(19) United States
(12) Patent Application Publication
(10) Pub. No.: US 2004/0127737 A1
(43) Pub. Date: Jul. 1, 2004

(54) Process for the preparation of carboxylic acid benzyl esters

(76) Inventors: Pieter Ooms, Krefeld (DE); Bernd-Ulrich Schenke, Bottrop (DE)

Correspondence Address: BAYER CHEMICALS CORPORATION, PATENT DEPARTMENT, 100 BAYER ROAD, PITTSBURGH, PA, 15205-9741, US

(21) Appl. No.: 10/664,307

(22) Filed: Sep. 17, 2003

(30) Foreign Application Priority Data
Sep. 18, 2002 (DE) 10243200.7

(51) Int. Cl. C07C 255/57; C07C 67/24

(52) U.S. Cl. 558/416; 560/20; 560/96

(57) ABSTRACT

Carboxylic acid benzyl esters can be prepared by reacting dibenzyl ethers with carboxylic anhydrides in the presence of acid, optionally applied to a support, as catalyst.

### BACKGROUND OF THE INVENTION

1. Field of the Invention

The present invention relates to a process for the preparation of carboxylic acid benzyl esters by reacting dibenzyl ethers with carboxylic anhydrides in the presence of acid as catalyst.

2. Brief Description of the Prior Art

Illustrative of the carboxylic acid benzyl esters is benzyl acetate, which is useful as the main component of jasmine oil, an important fragrance for the preparation of scent compositions and as starting material for the preparation of fruit ethers.

The preparation of benzyl acetate by esterifying benzyl alcohol with acetic acid has been known for a long time.

Benzyl acetate can also be prepared by reacting benzyl chloride with alkali metal acetates, if appropriate in the presence of phase transfer reagents (Wang et al., Chem. Eng. Commun., 100, p. 135 to 147 (1991)). A disadvantage of these processes is the formation of salts, which have to be disposed of, thus reducing their the cost-effectiveness.

DD-A5-286 577 describes the preparation of benzyl acetate by reacting dibenzyl ethers with acetic anhydride. A disadvantage of this process is the drastic reaction conditions (300° C./20 Mpa) and the only moderate yields.

The object was therefore to develop a process for the preparation of carboxylic acid benzyl esters starting from dibenzyl ethers which can be carried out under mild reaction conditions and leads to good yields in a cost-effective manner.

### SUMMARY OF THE INVENTION

In accordance with the foregoing, the present invention encompasses a process for the preparation of carboxylic acid benzyl esters of the formula

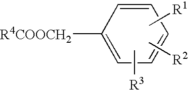

in which

R¹ to R³ are identical or different and are hydrogen, C₁-C₆-alkyl, C₁-C₆-alkoxy, C₁-C₆-haloalkyl, C₁-C₆-haloalkoxy, CN, CO(C₁-C₆-alkyl), NO₂ or halogen and

R⁴ is hydrogen, C₁-C₂₀-alkyl, C₂-C₂₀-alkenyl, C₇-C₁₄-aralkyl, C₆-C₁₂-aryl, C₁-C₆-haloalkyl, C₂-C₆-haloalkenyl or C₆-C₁₂-haloaryl,

from dibenzyl ethers, which is characterized in that dibenzyl ethers of the formula

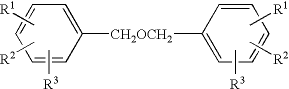

in which

R¹, R² and R³ have the meanings given above,

or mixtures of dibenzyl ethers and benzyl alcohols of the formula

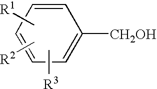

in which

R¹, R² and R³ have the meanings given above

are reacted with carboxylic anhydrides of the formula

(R⁴CO)₂O

in which

R⁴ has the meaning given above,

in the presence of at least one acid as catalyst, which may optionally be applied to a support.

As catalyst, one or more acids can be used. Preference is given to using one acid. The catalyst can be applied to one or more supports. Preference is given to using one support.

The process according to the invention can be carried out in a cost-effective manner and under mild reaction conditions.

### DETAILED DESCRIPTION OF THE INVENTION

The radicals R¹ to R³ generally have the following meanings:

alkyl generally means a straight-chain or branched hydrocarbon radical having 1 to 6, preferably 1 to 4, particularly preferably 1 or 2, carbon atoms. Examples which may be mentioned are methyl, ethyl, propyl, isopropyl, butyl, isobutyl, pentyl, isopentyl, hexyl and isohexyl. Preference is given to methyl, ethyl, propyl, isopropyl, butyl, pentyl and hexyl, in particular preference is given to methyl and ethyl.

Alkoxy generally means a straight-chain or branched alkoxy radical having 1 to 6, preferably 1 to 4, particularly preferably 1 or 2, carbon atoms. Examples which may be mentioned are methoxy, ethoxy, propoxy, isopropoxy, butoxy, isobutoxy, pentoxy, isopentoxy, hexoxy and isohexoxy. Preference is given to methoxy, ethoxy, propoxy, isopropoxy, butoxy, isobutoxy, pentoxy and hexoxy, in particular preference is given to methoxy and ethoxy.

Haloalkyl generally means a straight-chain or branched hydrocarbon radical having 1 to 6, preferably 1 to 4, particularly preferably 1 or 2, carbon atoms with 1 to 10, preferably 1 to 8, particularly preferably with 1 to 5, halogen atoms. For example, mention may be made of chloromethyl, fluoromethyl, difluoromethyl, trifluoromethyl, fluoroethyl, fluoropropyl and hexafluorobutyl. Preference is given to fluoromethyl, difluoromethyl, trifluoromethyl, fluoroethyl, fluoropropyl and hexafluorobutyl, and particular preference is given to fluoromethyl and trifluoromethyl.

Haloalkoxy generally means a straight-chain or branched alkoxy radical having 1 to 6, preferably 1 to 4, particularly preferably 1 or 2, carbon atoms with 1 to 10, preferably 1 to 8, particularly preferably with 1 to 5, halogen atoms. For example, mention may be made of chloromethoxy, fluoromethoxy, difluoromethoxy, trifluoromethoxy, fluoroethoxy, fluoropropoxy and hexafluorobutoxy. Preference is given to chloromethoxy, fluoromethoxy, trifluoromethoxy, fluoroethoxy, fluoropropoxy and hexafluorobutoxy, and particular preference is given to fluoromethoxy and trifluoromethoxy.

Halogen generally means fluorine, chlorine, bromine and iodine, preferably fluorine, chlorine and bromine, particularly fluorine and chlorine.

Very particularly preferred substituents for R¹ to R³ are hydrogen, methyl, trifluoromethyl, methoxy, fluorine or chlorine.

The following carboxylic acid benzyl esters can, for example, be prepared by the process according to the invention:

benzyl formate, benzyl acetate, benzyl chloroacetate, benzyl propionate, benzyl butyrate, benzyl pentanoate, benzyl hexanoate, benzyl heptanoate, benzyl octanoate, benzyl nonanoate, benzyl decanoate, benzyl undecanoate, benzyl dodecanoate, benzyl tridecanoate, benzyl tetradecanoate, benzyl pentadecanoate, benzyl hexadecanoate, benzyl heptadecanoate, benzyl octadecanoate, benzyl nonadecanoate, benzyl phenyl acetate, benzyl cinnamate, benzyl benzoate, benzyl 3-chlorobenzoate, benzyl 2-hydroxybenzoate, benzyl 3-hydroxybenzoate, benzyl 4-hydroxybenzoate, benzyl 3-chloro-2-hydroxybenzoate, benzyl 4-chloro-2-hydroxybenzoate, benzyl 5-chloro-2-hydroxybenzoate, benzyl 6-chloro-2-hydroxybenzoate, benzyl 3-bromo-2-hydroxybenzoate, benzyl 3-fluoro-2-hydroxybenzoate, benzyl 4-fluoro-2-hydroxybenzoate, benzyl 2-fluoro-3-hydroxybenzoate, benzyl 2-fluoro-4-hydroxybenzoate, benzyl 3-fluoro-2-hydroxybenzoate, benzyl 2-fluoro-5-hydroxybenzoate, benzyl 2-fluoro-6-hydroxybenzoate, benzyl 2-hydroxy-3-methylbenzoate, benzyl 2-hydroxy-4-methylbenzoate, benzyl 3-hydroxy-2-methylbenzoate, benzyl 4-hydroxy-2-methyl-benzoate, benzyl 2-fluoro-6-hydroxy-4-methoxybenzoate, benzyl 3-trifluoromethyl-2-hydroxybenzoate, benzyl 4-trifluoromethyl-2-hydroxybenzoate, benzyl 2-tri-fluoromethyl-3-hydroxybenzoate, benzyl 2-fluoroethyl-4-hydroxybenzoate and benzyl 4-fluorobutyl-2-hydroxybenzoate.

The dibenzyl ether used in the process according to the invention is an unsubstituted or substituted dibenzyl ether.

Particular preference is given to using an unsubstituted dibenzyl ether.

In a preferred embodiment of the invention, the dibenzyl ether is a substituted dibenzyl ether which carries one or more substituents from the series C₁-C₆-alkyl, C₁-C₆-alkoxy, CN, CO(C₁-C₆-alkyl), NO₂ or halogen.

In the process according to the invention, it is possible to use dibenzyl ether or dibenzyl ether/benzyl alcohol mixtures, as are produced, for example, in the preparation of benzyl alcohol from benzyl chloride. The content of dibenzyl ether can be 50 to 100%, preferably 60 to 99%, particularly preferably 70 to 98%.

The carboxylic anhydrides used in the process according to the invention are straight-chain and branched, saturated and unsaturated alkyl-, aralkyl- and arylcarboxylic anhydrides having 1 to 20 carbon atoms, such as, for example, acetic anhydride, propionic anhydride, isobutyric anhydride, valeric anhydride, isovaleric anhydride, caproic anhydride, heptanoic anhydride, caprylic anhydride, nonanoic anhydride, capric anhydride, undecanoic anhydride, lauric anhydride, tridecanoic anhydride, myristic anhydride, palmitic anhydride, stearic anhydride, oleic anhydride, linoleic anhydride, chloroacetic anhydride, linolenic anhydride, acrylic anhydride, meth-acrylic anhydride, cinnamic anhydride, phenylacetic anhydride, benzoic anhydride, or salicylic anhydride, and also mixed anhydrides, such as, for example, the mixed anhydride of formic acid and acetic acid. Preference is given to carboxylic anhydrides having 1 to 15 carbon atoms, particularly preferably 1 to 10 carbon atoms. Very particularly preferred carboxylic anhydrides are acetic anhydride, chloroacetic anhydride, propionic anhydride, butyric anhydride, isobutyric anhydride, valeric anhydride, hexanoic anhydride and benzoic anhydride.

In the process according to the invention, 1 to 50 equivalents, preferably 1 to 25 equivalents, particularly preferably 1 to 15 equivalents and very particularly preferably 1 to 10 equivalents, of carboxylic anhydride, based on dibenzyl ether, are used.

In the process according to the invention, it is possible to use homogeneous and also heterogeneous catalysts.

Suitable catalysts for the process according to the invention are inorganic acids, such as, for example, sulphur trioxide, sulphuric acid, hydrogen chloride, hydrogen bromide, hydrogen iodide, hydrofluoric acid, perchloric acid, chlorosulphonic acid or phosphoric acid, organic acids, such as, for example, trifluoroacetic acid, methane-sulphonic acid, ethanesulphonic acid, benzenesulphonic acid, 4-toluenesulphonic acid, chlorosulphonic acid or trifluoromethanesulphonic acid and Lewis acids, such as, for example, boron trifluoride, aluminium chloride, aluminium bromide, aluminium iodide, zinc chloride, tin chloride, titanium chloride or zirconium chloride, optionally applied to one or more, preferably one, support.

Preference is given to sulphur trioxide, sulphuric acid, trifluoromethanesulphonic acid, 4-toluenesulphonic acid, chlorosulphonic acid and boron trifluoride, particular preference is given to sulphur trioxide, sulphuric acid, trifluoromethanesulphonic acid, chlorosulphonic acid and boron trifluoride, optionally applied to a support.

Further suitable catalysts for the process according to the invention are heteropolyacids of the formula (I)

AₐX_{b}M_{c}O_{d} (I)

in which

A is protons and/or metal cations

x is P, Si, B, Ge, As, I, Se or Te

M is W, Mo, V or Cr

a is 3, 4, 5 or 6, such that the heteropolyacids or salts thereof are electroneutral

b is 1 or 2

c is 12 or 18 and

d is 40 or 62

optionally applied to one or more, preferably one, support.

Suitable cations A to be mentioned are, for example, cations of the alkali metals, such as lithium, sodium, potassium, rubidium or cesium, or cations of the metals manganese, nickel, cobalt, copper or lanthanum or protons.

Preferred heteropolyacids are phosphomolybdic acid, phosphotungstic acid, phosphovanadic acid, silicomolybdic acid, silicotungstic acid, silicovanadic acid, particularly preferred heteropolyacids are phosphomolybdic acid, phosphotungstic acid, silicomolybdic acid and silicotungstic acid, optionally applied to a support.

Preferred heteropolyacids are also those of the Keggin type, i.e. compounds of the formula (I) in which b is 1, c is 12 and d is 40, and those of the Dawson type, i.e. compounds of the formula (I) in which b is 2, c is 18 and d is 62. Particularly preferred compounds are A₃[PMo₁₂O₄₀], A₃[PW₁₂O₄₀], A₃[SiMo₁₂O₄₀] and A₃[SiW₁₂O₄₀].

Methods for the preparation of the heteropolyacids are known and are described, for example, in Römpp, Lexikon der Chemie Volume 3, 10^{th} Edition, Stuttgart/New York 1997, p. 1741; Chemical Reviews 98, 1998, 1ff or Catal. Rev. Sci. Eng. 37, 1995, 311ff.

In a preferred embodiment of the invention, the acids used are inorganic acids, organic acids or Lewis acids with a pH of from 1 to 6.

Suitable carriers for the process according to the invention are oxides or sulphates of elements of groups IIA (Group 2 according to IUPAC), for example magnesium, calcium or barium, III B (Group 3 according to IUPAC), for example scandium, yttrium or lanthanum, IV B (Group 4 according to IUPAC), for example titanium, zirconium or hafnium, V B (Group 5 according to IUPAC), for example niobium or tantallum, VII B (Group 7 according to IUPAC), for example manganese, VII (Group 8, 9 and 10 according to IUPAC), for example iron or nickel, III A (Group 13 according to IUPAC), for example Al and IV A (Group 14 according to IUPAC), for example silicon, germanium, tin or lead and carbon.

Examples to be mentioned are CaO, MgO, ZrO₂, TiO₂, HfO₂, SnO₂, Al₂O₃, SiO₂, Al₂O₃.SiO₂ (alumosilicates such as zeolites or phyllosilicates), Nb₂O₅, Ta₂O₅, Fe₂O₃, LaSO₄ or CaSO₄ and activated carbons.

Preference is given to CaO, MgO, ZrO₂, TiO₂, HfO₂, SnO₂, Al₂O₃.SiO₂, Al₂O₃, SiO₂, Nb₂O₅, Ta₂O₅, Fe₂O₃, LaSO₄ or CaSO₄, in particular preference is given to CaO, MgO, SnO₂, ZrO₂, TiO₂, HfO₂, Al₂O₃, SiO₂, Al₂O₃.SiO₂, Nb₂O₅ and Ta₂O₅.

As catalysts, sulphur trioxide, sulphuric acid and trifluoromethanesulphonic acid on activated carbon, CaO, MgO, ZrO₂, TiO₂, HfO₂, SnO₂, Al₂O₃, SiO₂, Al₂O₃.SiO₂ (alumosilicates such as zeolites or phyllosilicates), Nb₂O₅, Ta₂O₅, Fe₂O₃, LaSO₄ or CaSO₄ can preferably be used.

As catalysts, phosphomolybdic acid, phosphotungstic acid, phosphovanadic acid, silicomolybdic acid, silicotungstic acid or silicovanadic acid on activated carbon, CaO, MgO, ZrO₂, TiO₂, HfO₂, SnO₂, Al₂O₃, SiO₂, Al₂O₃.SiO₂ (alumosilicates such as zeolites or phyllosilicates), Nb₂₀₅, Ta₂O₅, Fe₂O₃, LaSO₄ or CaSO₄ can be used.

As catalysts, very particular preference is given to sulphated oxides (superacid) such as SO₃ on CaO, MgO, ZrO₂, TiO₂, HfO₂, SnO₂, Al₂O₃, SiO₂, Al₂O₃.SiO₂, Nb₂O₅, Ta₂O₅ or Fe₂O₃.

Methods for the preparation are well known and described, for example, in Applied Catalysis A, 146, 1996, p. 3 to 32, Catalysis Today 20, p. 219 to 256 (1994) and WO 00/64849).

The acids or their hydrates can be used as heterogeneous catalyst applied to a support, optionally calcined.

Suitable heterogeneous catalysts for the process according to the invention are preferably acidic ion exchangers, such as, for example, polymers carrying sulphonic acid groups, where the polymers may, for example, be polystyrenes, styrene-divinylbenzene copolymers or phenol-formaldehyde resins. Preferred acidic ion exchangers are sulphonylated polystyrenes, sulphonylated styrene-divinylbenzene copolymers or sulphonylated phenyl-formaldehyde resins, and very particular preference is given to sulphonylated polystyrenes.

In addition, particular preference is given to using fluorinated or perfluorinated polymers carrying sulphonic acid groups, such as, for example, fluorinated or perfluorinated sulphonylated polystyrenes, fluorinated or perfluorinated sulphonylated styrene-divenylbenzene copolymers or fluorinated or perfluorinated sulphonylated phenol-formaldehyde resins. Very particular preference is given to using fluorinated or perfluorinated sulphonylated polystyrenes.

The ion exchangers which carry sulphonic acid groups can be prepared by reacting polymers with sulphonating agents such as sulphuric acid or chlorosulphonic acid. The preparation is described, for example, in Encyclopedia of Polymer Science and Technology Vol. 7, Ed. N. M. Bikales, Interscience Publishers New York, 1967, p. 695 ff.

It is also possible to use mixtures of acidic ion exchangers.

Since it is known from Mastagli et al., C. r. 232, 1951, 1848-1849 that dibenzyl ether is converted in the presence of sulphonated phenol-formaldehyde resins to give toluene and benzaldehyde, the suitability of ion exchangers as catalysts in the process according to the invention is particularly surprising.

The acidic ion exchangers can be in spherical form and have particle sizes of from 0.3 to 3.0 mm in diameter. They can be of the gel type or macroporous. Their total capacity of acid functions in water-moist form with a water content of about 75 to 85% by weight is preferably 0.7 to 2.1 or 3.5 to 5 mval/ml of ion exchanger, based on 1 g of dry substance of ion exchanger. Suitable acidic ion exchangers are, for example, the products sold under the following registered trade names Lewatit®, Amberlite®, Dowex®, Duolite®, Nafion®, Permutit®, Chempro® or Imac®.

In the process according to the invention, the acidic ion exchangers are preferably used in dried form. The drying can be achieved by heat and/or vacuum. In addition, a drying can take place by washing with hydrophilic liquids such as, for example, the carboxylic anhydride used in the process, or by azeotropic distillation with organic solvents, such as toluene, xylene or methylene chloride.

The catalysts can be used, for example, as powders or mouldings and be separated off after the reaction by, for example, filtration, sedimentation or centrifugation.

In a preferred embodiment of the invention, the catalyst is used as fixed-bed catalyst.

In the event of an arrangement in the form of a fixed bed, the homogeneous catalysts are preferably applied to a support and used as mouldings, e.g. as spheres, cylinders, rods, hollow cylinders, rings, etc.

The heterogeneous catalysts are optionally dried by heat, optionally under reduced pressure, optionally by washing with hydrophilic organic liquids, such as, for example, the carboxylic anhydride used, or optionally by azeotropic distillation with organic liquids, such as toluene, xylene or methylene chloride.

When working in solution or suspension, the catalysts are used in stirred vessels in amounts of from 0.1 to 100% by weight, preferably from 0.5 to 90% by weight and particularly preferably from 1.0 to 80% by weight, based on dibenzyl ether.

In the case of a continuous procedure in countercurrent or cocurrent or in the trickle phase over a fixed-bed catalyst, space velocities of from 0.05 g to 5000 g of dibenzyl ether per litre of catalyst per hour, preferably of from 0.1 to 4000 g/1 h and particularly preferably of from 1.0 to 3000 g/1 h, are used.

In a preferred embodiment of the invention, one or more, preferably one, acid is used in an amount of from 0.5 to 100% by weight, based on the amount of dibenzyl ether, in the case of a dissolved or suspended catalyst, or with space velocities of from 1.0 to 3000 g of dibenzyl ether per litre of catalyst per hour in the case of an arrangement as a fixed-bed catalyst.

Preferably, the process according to the invention is carried out with intensive mixing of the reactants. Intensive mixing can be achieved in various ways known to the person skilled in the art, for example by stirrers, nozzles, baffles, static mixers, pumps, turbulent flows in narrow tubes or by ultrasound.

Such devices are described in more detail in Ullmann's Encyclopedia of Industrial Chemistry, 5^{th} Edition, Volume B, Unit Operations, Sections 25, 26, B4 pp. 569 to 570, Verlag Chemie, Weinheim 1988.

A preferred embodiment of the process according to the invention involves adding dibenzyl ether to a mixture or suspension of the heterogeneous catalyst and of the carboxylic anhydride and, when the reaction is complete, separating off the catalyst by, for example, filtration or centrifugation.

A further preferred embodiment is the cocurrent procedure in which dibenzyl ether and carboxylic anhydride are applied in cocurrent, for example from the top downwards onto a catalyst bed arranged in a tube, and carboxylic acid benzyl ester is stripped off at the bottom at the foot of the tube.

In a further preferred embodiment of the process according to the invention, this is carried out in the trickle phase and the catalyst is in the form of a fixed-bed catalyst. The catalyst bed is preferably in a vertical tubular reactor which preferably contains intermediate plates to improve distribution of the liquid stream and to improve wetting of the catalyst bed.

Both in the case of the suspended catalyst and also in the case of the fixed-bed process variants, work-up can be carried out by adding a water-immiscible solvent, preferably toluene, to the reaction products. After the organic phase, which comprises the crude carboxylic acid benzyl ester, has been separated off, it can, for example, be purified further by distillation.

The process according to the invention can be carried out batchwise, continuously or semicontinuously.

The temperature at which the process according to the invention is carried out is preferably 10 to 200, particularly preferably 25 to 190, very particularly preferably 30 to 180° C.

The pressure at which the process according to the invention is carried out is preferably 0.1 to 50 bar.

If the reaction is carried out above 140° C., it is necessary to work under elevated pressure corresponding to the vapour pressure. The required superatmospheric pressure is then at least equal to the vapour pressure of the reaction mixture. It can be up to 50 bar, preferably up to 25 bar.

The molar ratio of dibenzyl ether to carboxylic anhydride in the process according to the invention is preferably 1:1 to 1:50.

Where appropriate, the process according to the invention can be carried out under a customary protective gas, such as, for example, nitrogen, helium or argon.

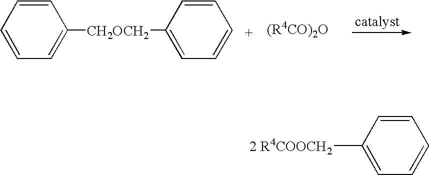

The process according to the invention gives carboxylic acid benzyl esters in good yields with a high conversion and good selectivity. The process according to the invention can be carried out simply without high expenditure on apparatus.

### EXAMPLES

The percentages in the examples below refer to the weight.

### Example 1

99.2 g (0.5 mol) of dibenzyl ether, 51.0 g (0.5 mol) of acetic anhydride and 1.0 g of conc. H₂SO₄ were heated at 80° C. in a flask fitted with baffles and paddle stirrer with vigorous stirring (250 rpm) and under nitrogen. After a reaction time of 7 hours, the mixture was cooled rapidly, and the organic phase was separated off following the addition of toluene and water and analyzed by gas chromatography. The reaction mixture comprised benzyl acetate and dibenzyl ether in the ratio 43:43.

### Example 2

Example 1 was repeated, but with a reaction temperature of 100° C. The reaction time was 7 hours. The reaction mixture comprised benzyl acetate and dibenzyl ether in the ratio 72:10.

### Example 3

Example 1 was repeated, but with a reaction temperature of 120° C. The reaction time was 5 hours. The reaction mixture comprised benzyl acetate and dibenzyl ether in the ratio 81:3.

### Example 4

Example 1 was repeated, but with 0.5 g of conc. H₂SO₄ and a reaction time of 7 hours. The reaction mixture comprised benzyl acetate and dibenzyl ether in the ratio 47:37.

### Example 5

Example 1 was repeated, but with 1.0 g of Lewatit SC 102 and a reaction temperature of 60° C. The reaction time was 5 hours. The reaction mixture comprised benzyl acetate and dibenzyl ether in the ratio 31:51.

### Example 6

Example 1 was repeated, but with 3.0 g of Lewatit SC 102 and a reaction temperature to of 80° C. The reaction time was 1 hour. The reaction mixture comprised benzyl acetate and dibenzyl ether in the ratio 75:4.

### Example 7

15 Example 1 was repeated, but with 89.0 g (0.5 mol) of chloroacetic anhydride and a reaction temperature of 100° C. The reaction time was 30 minutes. Benzyl chloroacetate was formed with a selectivity of 69%, based on the conversion of the dibenzyl ether.

### Example 8

At 120° C. with vigorous stirring (250 rpm) and under nitrogen, 99.2 g (0.5 mol) of Dibenzyl ether are added dropwise over the course of 50 min to a mixture of 53.6 g (0.525 mol) of acetic anhydride and 1.0 g of conc. H₂SO₄ in a flask fitted with baffles and paddle stirrer. After a reaction time of 5 hours, the mixture was cooled rapidly, and the organic phase was separated off following the addition of toluene and water and analyzed by gas chromatography. The reaction mixture comprised benzyl acetate and dibenzyl ether in the ratio 74:14.

### Example 9

Example 8 was repeated, but with a reaction temperature of 140° C. The reaction time was 2 hours. The reaction mixture comprised benzyl acetate and dibenzyl ether in the ratio 82:8.

### Example 10

Example 8 was repeated, but with 0.25 g of trifluoromethanesulphonic acid and a reaction temperature of 100° C. The reaction time was 1 hour. The reaction mixture comprised benzyl acetate and dibenzyl ether in the ratio 84:2.

### Example 11

Example 8 was repeated, but with 0.5 g of boron trifluoride diethyl etherate and a reaction temperature of 100° C. The reaction time was 2 hours. The reaction mixture comprised benzyl acetate and dibenzyl ether in the ratio 73:6.

### Example 12

Example 8 was repeated, but with 0.5 g of phosphotungstic acid and a reaction temperature of 80° C. The reaction time was 1 hour. The reaction mixture comprised benzyl acetate and dibenzyl ether in the ratio 71:6.

### Example 13

Example 8 was repeated, but with 2.0 g of a sulphated silica gel (75.0 g of SO₃/1 of SiO₂) and a reaction temperature of 100° C. The reaction time was 2 hours. The reaction mixture comprised benzyl acetate and dibenzyl ether in the ratio 32:60.

### Example 14

Example 8 was repeated, but with 2.0 g of a sulphated silica gel (20.0 g of SO₃/1 of SiO₂) and a reaction temperature of 100° C. The reaction time was 5 hours. The reaction mixture comprised benzyl acetate and dibenzyl ether in the ratio 29:60.

### Example 15

Example 8 was repeated, but with 2.0 g of a sulphated tantallum oxide (20.0 g of SO₃/1 of Ta₂O₅) and a reaction temperature of 100° C. The reaction time was 6 hours. The reaction mixture comprised benzyl acetate and dibenzyl ether in the ratio 35:52.

### Example 16

Example 8 was repeated, but with 2.0 g of a sulphated calcium sulphate (17.4 g of SO₃/1 of CaSO₄) and a reaction temperature of 100° C. The reaction time was 6 hours. The reaction mixture comprised benzyl acetate and dibenzyl ether in the ratio 43:46.

### Example 17

Example 8 was repeated, but with 2.0 g of a catalyst, prepared by applying 10 g of trifluoromethanesulphonic acid to 1000 ml of silica gel. The reaction time was 5 hours. The reaction mixture comprised benzyl acetate and dibenzyl ether in the ratio 28:60.

### Example 18

Example 8 was repeated, but with 2.0 g of a catalyst prepared by applying 75.0 g of phosphotungstic acid to 1000 ml of TiO₂ and a reaction temperature of 100° C. The reaction time was 2 hours. The reaction mixture comprised benzyl acetate and dibenzyl ether in the ratio 55:23.

### Example 19

Example 8 was repeated, but with 68.3 g (0.525 mol) of propionic anhydride. The reaction time was 6 hours. The reaction mixture comprised benzyl propionate and dibenzyl ether in the ratio 53:34.

### Example 20

Example 8 was repeated, but with 118.8 g (0.525 mol) of benzoic anhydride. The reaction time was 3 hours. The reaction mixture comprised benzyl benzoate and dibenzyl ether in the ratio 78:4.

### Example 21 (Work-up)

Example 9 was repeated and worked up after a run time of 3 hours. Following neutralization and distillative separation of the reaction mixture, 91 g (61%) of benzyl acetate with a purity of 99.1% are isolated at 107-109° C./33 mbar. The forerunning still comprises 14 g (9%) of benzyl acetate, and the residue comprises 13 g (9%) of benzyl acetate and 5 g (5%) of dibenzyl ether. Yield 79% (83% based on reacted dibenzyl ether).

1. Process for the preparation of carboxylic acid benzyl esters of the formula 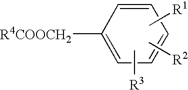
in which
R¹ to R³ are identical or different and are C₁-C₆-alkyl, C₁-C₆-alkoxy, Cₗ-C₆-haloalkyl, C₁-C₆-haloalkoxy, CN, CO(C₁-C₆-alkyl), NO₂ or halogen and
R⁴ is hydrogen, C₁-C₂₀-alkyl, C₂-C₂₀-alkenyl, C₇-C₁₄-aralkyl, C₆-C₁₂-aryl, C₁-C₆-haloalkyl, C₂-C₆-haloalkenyl or C₆-C₁₂-haloaryl,
from dibenzyl ethers, comprising:
reacting dibenzyl ethers of the formula 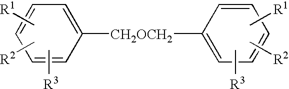
in which
R¹, R² and R³ have the meanings given above,
or mixtures of dibenzyl ethers and benzyl alcohols of the formula 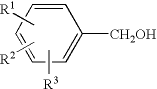
in which
R¹, R² and R³ have the meanings given above
with carboxylic anhydrides of the formula
(R⁴CO)₂O
in which
R⁴ has the meaning given above,
in the presence of at least one acid as catalyst, which may optionally be applied to a support.

2. Process according to claim 1, characterized in that the acids are inorganic acids, organic acids or Lewis acids with a pH of from 1 to 6.

3. Process according to claim 1, characterized in that the acids are sulphur trioxide, sulphuric acid, hydrogen chloride, hydrogen bromide, hydrogen iodide, hydrofluoric acid, perchloric acid, chlorosulphonic acid, phosphoric acid, trifluoroacetic acid, methanesulphonic acid, ethanesulphonic acid, benzenesulphonic acid, 4-toluenesulphonic acid, boron trifluoride, aluminium chloride, aluminium bromide, aluminium iodide, zinc chloride, tin chloride, titanium chloride or zirconium chloride, optionally applied to one or more supports.

4. Process according to claim 1, characterized in that heteropolyacids of the formula (I) are used
AₐX_{b}M_{c}O_{d} (I)
in which
A is protons and/or metal cations
x is P, Si, B, Ge, As, I, Se or Te
M is W, Mo, V or Cr
a is 3, 4, 5 or 6, so that the heteropolyacids or salts thereof are electroneutral
b is 1 or 2
c is 12 or 18 and
d is 40 or 62,
optionally applied to one or more supports.

5. Process according to claim 4, characterized in that A is a cation which is hydrogen, lithium, sodium, potassium, rubidium, cesium, manganese, nickel, cobalt, copper or lanthanum.

6. Process according to claim 4, characterized in that the heteropolyacids used are phosphomolybdic acid, phosphotungstic acid, phosphovanadic acid, silicomolybdic acid, silicotungstic acid or silicovanadic acid, optionally applied to one or more supports.

7. Process according to claim 1, characterized in that the acid used is an acidic ion exchanger.

8. Process according to claim 7, characterized in that the acid used is a polymer carrying sulphonic acid groups.

9. Process according to claim 1, characterized in that the reaction is carried out at a temperature of from 10 to 200° C.

10. Process according to claim 1, characterized in that the acid used is a sulphated oxide.
